(19) United States
(12) Patent Application Publication
(10) Pub. No.: US 2008/0312201 A1
(43) Pub. Date: Dec. 18, 2008

(54) Reduced Toxicity Methotrexate Formulations and Methods for Using the Same

(76) Inventor: Patrick Fogarty, Santa Cruz, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/662,375

(22) PCT Filed: Sep. 8, 2005
(86) PCT No.: PCT/US05/32046

§ 371 Date: May 12, 2008

Related U.S. Application Data
(60) Provisional application No. 60/609,046, filed on Sep. 10, 2004.

(51) Int. Cl. A61K 31/58; A61K 31/519; A61P 35/00; A61K 31/295

(52) U.S. Cl. 514/176; 514/262.1; 514/184

(57) ABSTRACT

Methods of using methotrexate (MTX) active agents in which reduced host toxicity is observed are provided. In the subject methods, an effective amount of an MTX active agent is administered to the host in conjunction with the administration of an MTX toxicity reducing agent of the present invention. Also provided are compositions for use in practicing the subject methods, e.g., pharmaceutical compositions having reduced toxicity, in which the MTX active agent is combined with an MTX toxicity reducing agent that reduces the level of undesired MTX toxicity while maintaining an effective MTX anti-proliferative activity. Also provided are methods of using the subject methods and compositions in the treatment of a variety of different disease conditions.

### CROSS-REFERENCE TO RELATED APPLICATIONS

Pursuant to 35 U.S.C. § 119 (e), this application claims priority to the filing date of the U.S. Provisional Patent Application Ser. No. 60/609,046 filed Sep. 10, 2004; the disclosure of which is herein incorporated by reference.

### INTRODUCTION

### Background of the Invention

Within the cell, important molecules called tetrahydrofolates (THF) power the life-sustaining processes of DNA synthesis, replication and repair by coenzymatically providing substrates necessary for these processes. THF are biosynthesized intracellularly through reduction of folic acid or other dihydrofolate intermediates by the enzyme dihydrofolate reductase (DHFR). The pteridine compound, methotrexate (MTX; N-[4-[[(2,4-diamino-6-pteridinyl methyl]methylamino]benzoyl]-L-glutamicacid), is structurally quite similar to folic acid. As a result of this structural similarity, MTX can bind to active sites on DHFR, and, through competitive inhibition, block the formation of THF needed in the biosynthesis of DNA and RNA.

This ability of MTX to inhibit nucleic acid synthesis has been exploited in the treatment of aberrant cell growth. In particular, since many malignant cells proliferate more rapidly than normal cells, and since actively proliferating cells are more sensitive to the effect of MTX, in many cases, MTX can be used to selectively impair cancerous cell growth without damaging normal cell growth. As a result of its effectiveness against rapidly proliferating cells, MTX is one of the most widely used anticancer agents. For example, MTX is employed in the treatment of neoplastic diseases such as gestational choriocarcinoma, chorioadenoma destruens, hydatidiform mole, acute lymphocytic leukemia, breast cancer, epidermoid cancers of the head and neck, advanced mycosis fungoides, lung cancer, and non-Hodgkins lymphomas (Physicians Desk Reference, 45th ed., Medical Economical Co., Inc., 1185-89 (Des Moines, Iowa (1991)). Moreover, MTX is an effective immunosuppressive agent, with utility in the prevention of the graft-versus-host reaction that can result from tissue transplants, as well as in the management of inflammatory diseases. Consequently, MTX can be employed in the treatment of severe and disabling psoriasis and rheumatoid arthritis (Hoffmeister, The American Journal of Medicine, 30, 69-73 (1983); Jaffe, Arthritis and Rheumatism, 31, 299 (1988)).

The numerous patents that have been issued disclosing MTX and MTX analogs, methods of synthesizing MTX or analogs thereof, and uses for MTX attest to the significance of MTX in treatment of aberrant cell growth. For example, U.S. Pat. No. 2,512,572 covers the active agent MTX, and U.S. Pat. Nos. 3,892,801, 3,989,703, 4,057,548, 4,067,867, 4,079,056, 4,080,325, 4,136,101,4,224,446, 4,306,064, 4,374,987, 4,421,913, and 4,767,859 claim methods for preparing MTX or potential intermediates in the synthesis of MTX. Other patents disclose labeled analogs of MTX, such as U.S. Pat. Nos. 3,981,983, 4,043,759, 4,093,607, 4,279,992, 4,376,767, 4,401,592, 4,489,065, 4,622,218, 4,625,014, 4,638,045, 4,671,958, 4,699,784, 4,785,080, 4,816,395, 4,886,780, 4,918;165, 4,925,662, 4,939,240, 4,983,586, 4,997,913, 5,024,998, 5,028,697, 5,030,719, 5,057,313, 5,059,413, 5,082,928, 5,106,950, and 5,108,987, wherein MTX is bound to a radionucleotide or fluorescent label, amino acid, polypeptide, transferrin or ceruloplasmin, chondroitin or chondroitin sulfate, antibody, or binding partner for a specific cell-surface receptor of target cells for use in assays of MTX, in timed-release of MTX, as toxins selective for cancer cells, or to facilitate transport of MTX across membranes or in vivo barriers. Of the numerous patents issued disclosing methods of using MTX, a variety of patents such as U.S. Pat. Nos. 4,106,488, 4,558,690, and 4,662,359 disclose methods of using MTX to treat cancer. Additionally, U.S. Pat. Nos. 4,396,601 and 4,497,796 describe the use of MTX to select cells that have been transfected with vectors containing a DHFR selectable marker, and U.S. Pat. No. 5,043,270 discloses the use of MTX to select for or assess gene amplification events. The basis for these two latter approaches is that an increase in the number of copies of the DHFR gene within a cell correspondingly increases resistance to MTX.

Despite the broad utility and utilization of MTX, treatment with this agent involves a strong risk to the patient. Since MTX interferes with cell replication and division, actively proliferating non-malignant tissues such as bone marrow and intestinal mucosa are more sensitive to MTX and may demonstrate impaired growth due to treatment. More importantly, MTX is associated with renal and hepatic toxicity when applied in the “high dose regimen” that is typically required for maximum efficiency (Barak et al., J. American Coll. Nutr., 3, 93-96 (1984)).

To alleviate MTX-induced toxicity, high dose MTX therapy can be administered in conjunction with citrovorum factor as a “rescue” agent for normal cells (Christenson et al., J. Clin. Oncol., 6, 797-801 (1988)). While citrovorum factor rescue reduces MTX toxicity to non-malignant cells, it does not solve the problem of renal and hepatic impairment due to the formation of 7-OH-MTX.

Because of the undisputed value of MTX in therapy and research, attempts have been made to increase the effectiveness of MTX and decrease the problems attendant with its use. Many investigators have modified the structure of MTX in attempts to synthesize more potent MTX derivatives. The most effective derivatives include aminopterin, which possesses a hydrogen instead of a methyl group at position N-10, and 4-amino derivatives with halogen substitution on the para-aminobenzoic moiety, such as dichloromethotrexate (Frei et al., Clin. Pharmacol. and Therap., 6, 160-71 (1965)). Additional MTX derivatives have been synthesized by: (i) preparing ester derivatives of the glutamyl moiety, (ii) replacing the glutamic acid with amino acids and peptides, (iii) adding a methyl group at the 7-position, (iv) poly-(L-lysine) conjugation, and (v) substituting the gamma amides (Rosowsky and Yu, J. Med. Chem., 21, 170-75 (1978); Rosowsky et al., J. Med. Chem., 21, 380-86 (1978); Chaykovsky et al., J. Med. Chem., 18, 909-12 (1975); Rosowsky and Chen, J. Med. Chem., 17, 1308-11 (1974)). More recent modification attempts include the synthesis of lysine and ornithine derivatives of MTX (Kempton et al., J. Med Chem., 25, 475-477 (1982); Patil et al., J. Med. Chem., 32, 1559-65 (1989)). These attempts to improve the efficacy of MTX have not yet proven entirely successful. Whereas some of the MTX derivatives, such as 7-methyl substituted MTX (Rosowsky and Chen, J. Med. Chem., 17, 1308-11 (1974)), demonstrate impaired antifolate antagonism, others, such as 3′,5′-difluoro MTX, demonstrate little or no increase in biological activity as compared with MTX (Tomcuf, J. Organic Chem., 26, 3351 (1961)). Still other derivatives, like the 2′ and 3′ monofluorinated derivatives of aminopterin, appear promising, but animal studies remain to be performed (Henkin and Washtien, J. Med. Chem., 26, 1193-1196 (1983)). Similarly, 7,8-dihydro-8-methyl-MTX has been prepared, but the biological properties of this and other compounds remain to be fully investigated (Chaykovsky, J. Org. Chem., 40 (1), 145-146 (1975)).

While the above approaches to reducing unwanted MTX toxicity have met with some success, there is still a need for improvement and advances in this area. The present invention addresses this need.

### Relevant Literature

United States patents of interest include: U.S. Pat. Nos. 2,512,572; 3,892,801; 3,981,983; 3,989,703; 4,043,759; 4,057,548; 4,067,867; 4,079,056; 4,080,325; 4,093,607; 4,136,101; 4,224,446; 4,279,992; 4,306,064; 4,374,987; 4,401,592; 4,421,913; 4,489,065; 4,622,218; 4,625,014; 4,638,045; 4,671,958; 4,699,784; 4,767,859; 4,785,080; 4,816,395; 4,886,780; 4,918,165; 4,925,662; 4,939,240; 4,983,586; 4,997,913; 5,024,998; 5,028,697; 5,030,719; 5,057,313; 5,059,413; 5,082,928; 5,106,950; and 5,108,987.

### BRIEF SUMMARY OF THE INVENTION

Methods of using methotrexate (MTX) active agents in which reduced host toxicity is observed are provided. In the subject methods, an effective amount of an MTX active agent is administered to the host in conjunction with the administration of an MTX toxicity reducing agent of the present invention. Also provided are compositions for use in practicing the subject methods, e.g., MTX pharmaceutical compositions having reduced toxicity and kits that include the same. The subject methods and compositions find use in a variety of different applications, including the treatment of a variety of different disease conditions.

### DESCRIPTION OF THE SPECIFIC EMBODIMENTS

Methods of using MTX active agents in which reduced host toxicity is observed are provided. In the subject methods, an effective amount of an MTX active agent is administered to the host in conjunction with the administration of an MTX toxicity reducing agent of the present invention. Also provided are compositions for use in practicing the subject methods, e.g., MTX pharmaceutical compositions having reduced toxicity and kits that include the same. The subject methods and compositions find use in a variety of different applications, including the treatment of a variety of different disease conditions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, the subject methods are described first in greater detail, followed by a review of the various compositions, e.g., formulations and kits, that may find use in the subject methods, as well as a discussion of various representative applications in which the subject methods and compositions find use.

### Methods

As summarized above, methods of administering an MTX active agent to a host in need thereof, e.g., for the treatment of a host suffering from disease or condition treatable by an MTX active agent (as described in greater detail below), are provided. A feature of the subject methods is that the MTX active agent of interest to be administered is administered in conjunction with an MTX toxicity reducing agent. By “in conjunction with” is meant that the MTX toxicity reducing agent is administered anywhere from simultaneously to up to 5 hours or more, e.g., 10 hours, 15 hours, 20 hours or more, prior to or after the MTX active agent. Thus, the toxicity reducing agent and the MTX active agent may be administered either: (a) sequentially, with the toxicity reducing agent being administered prior to or after the MTX active agent or (b) simultaneously, with the toxicity reducing agent being administered to the subject at the same time as the MTX active agent. Where the toxicity reducing agent is administered simultaneously with the MTX active agent, the two components may be administered as either a single, combined composition or as two distinct compositions that are simultaneously administered to the host.

In the subject methods, an effective amount of an MTX active agent is administered to a host in need thereof in combination with an effective amount of an MTX toxicity reducing agent. By MTX active agent is meant methotrexate or an analogue/derivative thereof. Methotrexate and analogues/derivatives thereof which may be present in the subject compositions include, but are not limited to those compounds described in U.S. Pat. Nos. 2,512,572; 3,892,801; 3,989,703; 4,057,548; 4,067,867; 4,079,056; 4,080,325; 4,136,101; 4,224,446; 4,306,064; 4,374,987; 4,421,913; 4,767,859; 3,981,983; 4,043,759; 4,093,607; 4,279,992; 4,376,767; 4,401,592; 4,489,065; 4,622,218; 4,625,014; 4,638,045; 4,671,958; 4,699,784; 4,785,080; 4,816,395; 4,886,780; 4,918,165; 4,925,662; 4,939,240; 4,983,586; 4,997,913; 5,024,998; 5,028,697; 5,030,719; 5,057,313; 5,059,413; 5,082,928; 5,106,950; 5,108,987; 4,106,488; 4,558,690; 4,662,359; 4,396,601; 4,497,796; 5,043,270; 5,166,149; 5,292,731; 5,354,753; 5,382,582; 5,698,556; 5,728,692; and 5,958,928; the disclosures of which are herein incorporated by reference.

MTX active agents of the present invention include MTX and any analogues/derivatives thereof whose toxicity is reduced when administered in conjunction with a toxicity reducing agent according to the subject invention. Whether or not a given MTX active agent is suitable for use according to the present invention can be readily determined using assays employed in the experimental section, below. Generally, an MTX active agent is suitable for use in the subject methods if its toxicity is reduced by at least about 2 to 10-fold, usually by at least about 50-fold and more usually by at least about 100-fold, as determined using the Drosophila assay described in the Experimental section, below. In certain embodiments, the MTX active agent is one that reduces the occurrence and/or intensity of observable toxic side effects as observed in the mouse assay described in the experimental section below.

By MTX toxicity reducing agent is meant an agent that reduces unwanted toxicity of an MTX active agent. Toxicity reducing agents of interest are those agents that reduce the toxicity of an MTX active agent by at least about 2 to 10-fold, usually by at least about 50-fold and more usually by at least about 100-fold, as determined using the Drosophila assay described in the Experimental section, below. In certain embodiments, the toxicity reducing agents of interest are those that reduce the occurrence and/or intensity of observable toxic side effects of a given MTX active agent, as observed in the mouse assay described in the experimental section below.

In representative embodiments, the toxicity reducing agents of interest are small organic compounds, typically having a mass of from about 50 to 2,500 hundred daltons, such as from about 100 to about 1,500 daltons. Compounds of interest may include functional groups necessary for structural interaction with proteins, particularly hydrogen bonding, and in representative embodiments typically include at least an amine, carbonyl, hydroxyl or carboxyl group, such as at least two of the functional chemical groups. The compounds may include cyclical carbon or heterocyclic structures and/or aromatic or polyaromatic structures substituted with one or more of the above functional groups. Compounds of interest may also include biomolecules including, but not limited to: peptides, saccharides, fatty acids, steroids, purines, pyrimidines, derivatives, structural analogs or combinations thereof.

Compounds of interest in representative embodiments are those obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling.

As such, in certain embodiments, the compounds include one or more ring structures, which may or may not be fused, may or may not be aromatic, and may or may not include one or more heteroatoms, e.g., N, S or O. In certain embodiments, the compounds of interest do not include any ring structures.

Representative toxicity reducing agents include, but are not limited to:

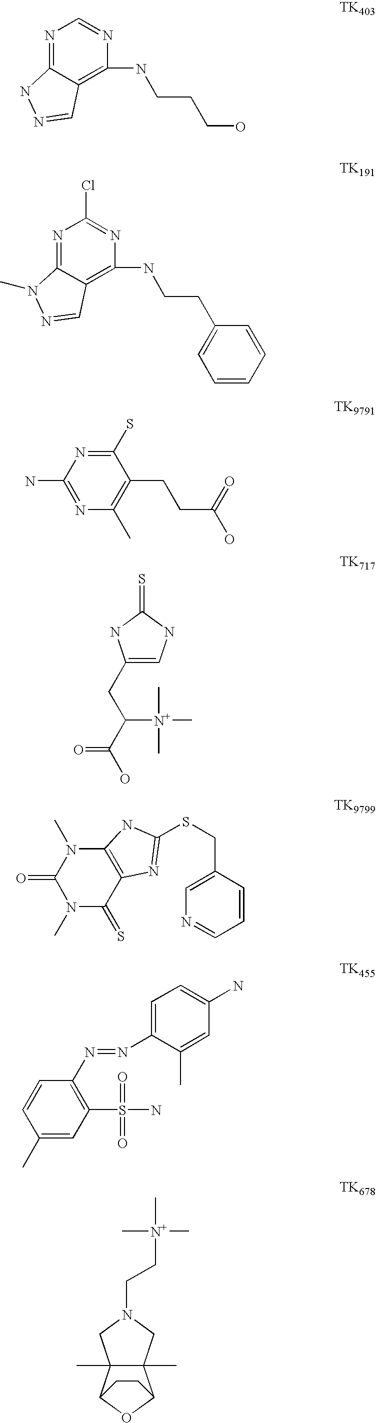

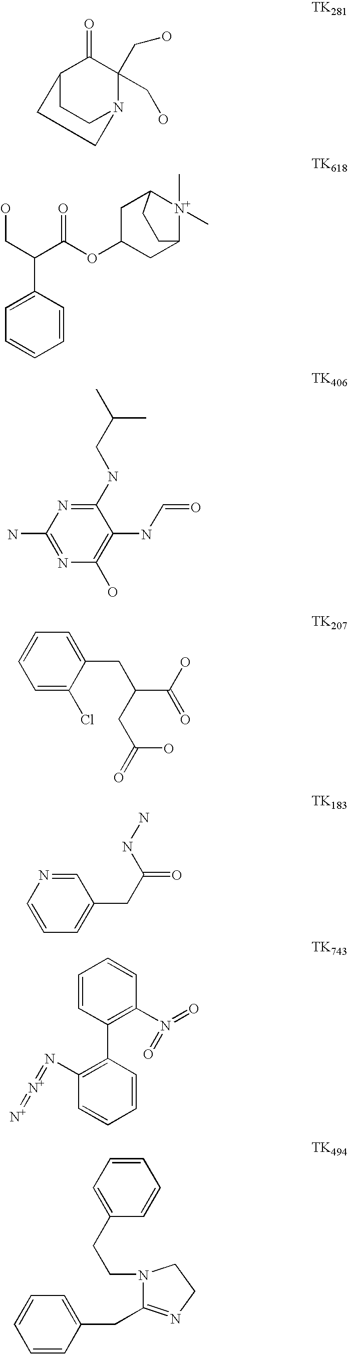

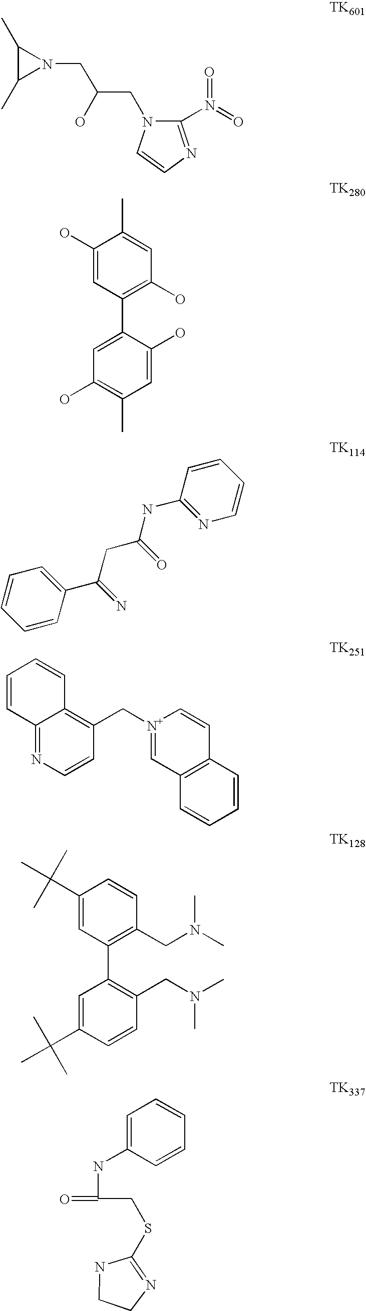

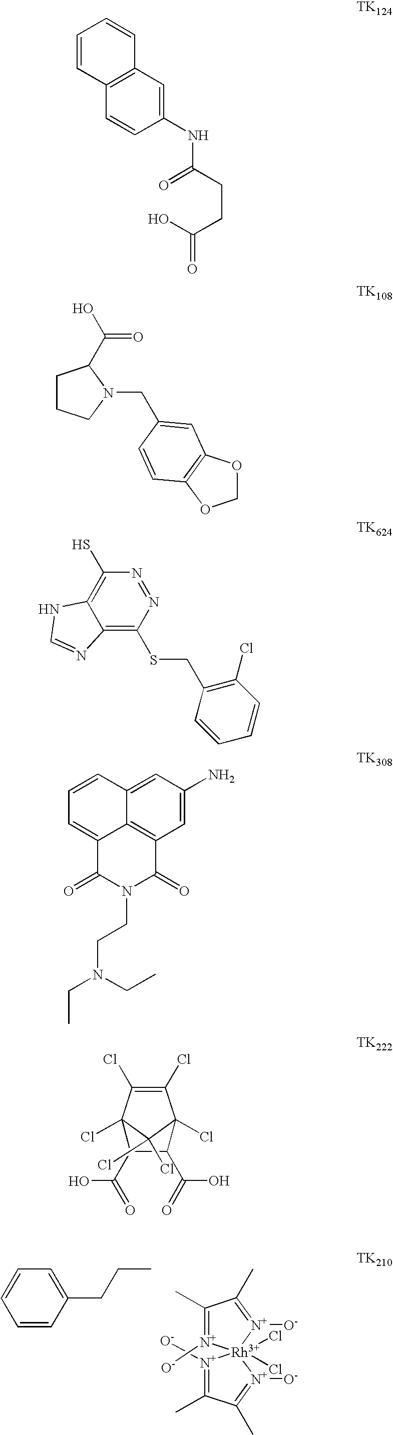

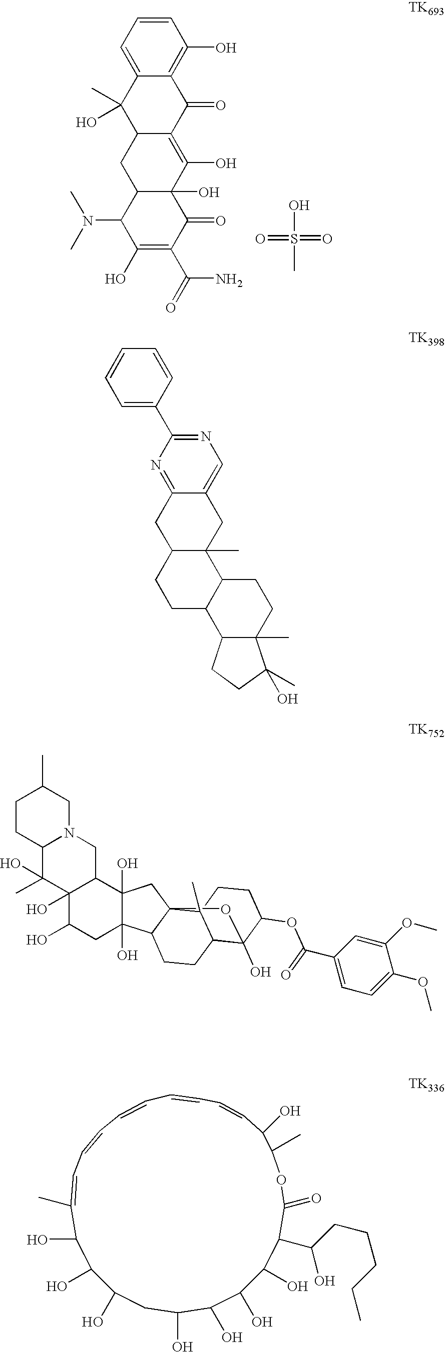

Also of particular interest are analogues/derivatives of the above compounds, where representative such analogues/derivatives have MTX reducing toxicity, such that MTX toxicity is reduced when the compounds are administered in conjunction with MTX according to the subject invention.

As indicated above, an effective amount of toxicity reducing agent is employed in the subject methods. In certain embodiments, the amount of toxicity reducing agent employed is not more than about the amount of the MTX active agent employed. In certain embodiments, an amount is an amount that is less than equimolar to the amount of MTX active agent that is administered. Typically, the amount of toxicity reducing agent that is administered is less than about 75%, less than about 50%, less then about 25% and many embodiments less than about 15%, less than about 10% and even less than about 5% or 1% than the amount of MTX active agent. In other embodiments, the effective amount is the same as the amount of the active agent, and in certain embodiments the effective amount is an amount that is more than the amount of the MTX active agent. Effective amounts can readily be determined empirically using the data provided in the experimental section, below.

### Formulations

Also provided are formulations that find use in the practicing the subject invention, where the formulations include at least one of the MTX active and the MTX toxicity reducing agent in a pharmaceutically acceptable delivery vehicle, such that in certain embodiments, a first formulation of MTX active agent and a second formulation of a MTX toxicity reducing agent are provided, while in other embodiments a single formulation that includes both the MTX active agent and the MTX toxicity reducing agent are provided.

In certain embodiments of interest, the MTX active agent and the toxicity reducing agent are administered as a single pharmaceutical formulation, that, in addition to including an effective amount of the active agent and toxicity reducing agent, includes other suitable compounds and carriers, and also may be used in combination with other active agents. The present invention, therefore, also includes pharmaceutical compositions comprising pharmaceutically acceptable excipients. The pharmaceutically acceptable excipients include, for example, any suitable vehicles, adjuvants, carriers or diluents, and are readily available to the public. The pharmaceutical compositions of the present invention may further contain other active agents as are well known in the art.

One skilled in the art will appreciate that a variety of suitable methods of administering a formulation of the present invention to a subject or host, e.g., patient, in need thereof, are available, and, although more than one route can be used to administer a particular formulation, a particular route can provide a more immediate and more effective reaction than another route. Pharmaceutically acceptable excipients are also well-known to those who are skilled in the art, and are readily available. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are known in the art to be appropriate.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Preferred dosages for a given compound are readily determinable by those of skill in the art by a variety of means.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to effect the desired growth inhibitory or immunosuppressive response. In the treatment of some individuals with the compounds of the present invention, it may be desirable to use a high dose regimen in conjunction with a rescue agent for non-malignant cells. In such treatment, any agent capable of rescue of non-malignant cells can be employed, such as citrovorum factor, folate derivatives, or leucovorin. Such rescue agents are well known to those of ordinary skill in the art. A rescue agent is preferred which does not interfere with the ability of the present inventive compounds to modulate cellular function.

### Utility

The subject methods find use in a variety of application, where in many applications the methods are modulating at least one cellular function, such as DHFR mediation of DNA synthesis or repair. In this respect, the subject methods and composition find use in known applications of MTX, such as in treating diseases or disorders that are capable of being treated using MTX. Use of the subject compositions of the present invention is of particular utility in, for example, in the treatment of diseases and disorders including but not limited to cancer, psoriasis, rheumatoid arthritis, and tissue-graft rejection, as well as in conditions requiring immunosuppressive agents. In these capacities, use of the present inventive compositions will result in a reduced unwanted toxicity while a retention of desired MTX activity.

As such, the subject methods and compositions find use in therapeutic applications in which MTX administration is indicated. A representative therapeutic application is the treatment of cellular proliferative disease conditions, e.g., cancers and related conditions characterized by abnormal cellular proliferation concomitant. Such disease conditions include cancer/neoplastic diseases and other diseases characterized by the presence of unwanted cellular proliferation, e.g., hyperplasias, and the like.

By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The subject methods find use in, among other applications, the treatment of cellular proliferative disease conditions, including neoplastic disease conditions, i.e., cancers. In such applications, an effective amount of the MTX active agent and MTX toxicity reducing agent, is administered to the subject in need thereof. Treatment is used broadly as defined above, e.g., to include at least an amelioration in one or more of the symptoms of the disease, as well as a complete cessation thereof, as well as a reversal and/or complete removal of the disease condition, e.g., cure.

There are many disorders associated with a dysregulation of cellular proliferation, i.e., cellular hyperproliferative disorders. The conditions of interest include, but are not limited to, the following conditions.

The subject methods may be employed in the treatment of a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Diseases where there is hyperproliferation and tissue remodelling or repair of reproductive tissue, e.g. uterine, testicular and ovarian carcinomas, endometriosis, squamous and glandular epithelial carcinomas of the cervix, etc. are reduced in cell number by administration of the subject compounds

Tumors of interest for treatment include carcinomas, e.g. colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g. neuroblastoma, gliomas, etc.; hematological malignancies, e.g. childhood acute leukemia, acute myelogenous leukemias, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue remodelling and repair. For example, the chronic skin inflammation of psoriasis is associated with hyperplastic epidermal keratinocytes as well as infiltrating mononuclear cells, including CD4+ memory T cells, neutrophils and macrophages.

The methods of the present invention can provide a highly general method of treating many-if not most-malignancies, including tumors derived from cells selected from skin, connective tissue, adipose, breast, lung, stomach, pancreas, ovary, cervix, uterus, kidney, bladder, colon, prostate, central nervous system (CNS), retina and blood, and the like. Representative cancers of interest include, but are not limited to: Head/Neck and Lung tissue (e.g., Head and neck squamous cell carcinoma, Non-small cell lung carcinoma, Small cell lung carcinoma) Gastrointestinal tract and pancreas (e.g., Gastric carcinoma, Colorectal adenoma, Colorectal carcinoma, Pancreatic carcinoma); Hepatic tissue (e.g., Hepatocellular carcinoma), Kidney/urinary tract (e.g., Dysplastic urothelium, Bladder carcinoma, Renal carcinoma, Wilms tumor) Breast (e.g., Breast carcinoma ); Neural tissue (e.g., Retinoblastoma, Oligodendroglioma, Neuroblastoma, Meningioma malignant; Skin (e.g., Normal epidermis, Squamous cell carcinoma, Basal cell carcinoma, Melanoma, etc.); Hematological tissues (e.g., Lymphoma, CML chronic myeloid leukemia, APL acute promyelocytic leukemia, ALL acute lymphoblastic leukemia, acute myeloid leukemia, etc.); and the like.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to effect the desired growth inhibitory or immunosuppressive response, particularly unmodified methotrexate. The preferred dosage is the amount which results in the inhibition of DHFR, without significant side effects. In proper doses and with suitable administration of certain compounds, the present invention provides for a wide range of intracellular effects, e.g., from partial inhibition to essentially complete inhibition of DHFR. This is especially important in the context of the present invention, as this differential inhibition can potentially be used to discriminate between cancer cells and highly proliferative non-malignant cells.

In the treatment of some individuals with the compounds of the present invention, it may be desirable to use a high dose regimen in conjunction with a rescue agent for non-malignant cells. In such treatment, any agent capable of rescue of non-malignant cells can be employed, such as citrovorum factor, folate derivatives, or leucovorin. Such rescue agents are well known to those of ordinary skill in the art. A rescue agent is preferred which does not interfere with the ability of the present inventive compounds to modulate cellular function.

Particular applications in which the subject methods and compositions find use include those described in U.S. Pat. Nos. 2,512,572; 3,892,801; 3,989,703; 4,057,548; 4,067,867; 4,079,056; 4,080,325; 4,136,101; 4,224,446; 4,306,064; 4,374,987; 4,421,913; 4,767,859; 3,981,983; 4,043,759; 4,093,607; 4,279,992; 4,376,767; 4,401,592; 4,489,065; 4,622,218; 4,625,014; 4,638,045; 4,671,958; 4,699,784; 4,785,080; 4,816,395; 4,886,780; 4,918,165; 4,925,662; 4,939,240; 4,983,586; 4,997,913; 5,024,998; 5,028,697; 5,030,719; 5,057,313; 5,059,413; 5,082,928; 5,106,950; 5,108,987; 4,106,488; 4,558,690; 4,662,359; 4,396,601; 4,497,796; 5,043,270; 5,166,149; 5,292,731; 5,354,753; 5,382,582; 5,698,556; 5,728,692; and 5,958,928; the disclosures of which are herein incorporated by reference.

### Kits

Kits with formulations used in the subject methods, are provided. Conveniently, the formulations may be provided in a unit dosage format, which formats are known in the art.

In such kits, in addition to the containers containing the formulation(s), e.g. unit doses, is an informational package insert describing the use of the subject formulations in the methods of the subject invention, i.e. instructions for using the subject unit doses to treat cellular proliferative disease conditions.

These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples further illustrate the present invention and should not be construed as in any way limiting its scope.

### EXPERIMENTAL

### I. Lethal Dose (LD) Curve Data

The LD curve in fruit flies was generated for methotrexate. The data to generate the curve was obtained by:
Mixing a specific concentration of chemical into the food and water supply of the fruit flies, then 50 wild-type embryos are added to the assay. The LD value for this concentration was calculated by 100−(2×(number of alive flies)). The LD curve was generated by repeating this method over a concentration range. For example, the concentration range tested for methotrexate was 0.01 mM to 5 mM. The LD 98 was identified for methotrexate was 0.5 mM. The LD98 was used as a stringent level for identifying additive chemicals that reduce the toxicity. This stringent level of toxicity is key for several reasons: 1) The high toxicity dose turns even mild toxic side effects into significant barriers for the flies to survive. At the LD98 concentration of methotrexate, all of the toxic mechanisms are orders of magnitude above that observed at physiological treatment doses. At the LD98 dose, suppressing any one toxicity side effect will not enable significant survival of the flies. An additive that enables significant survival is more likely able to reduce all toxic side effects of methotrexate.

### II. Additive Identification Results

A small molecule library containing 10,000 diverse structures was screened for additive compounds for methotrexate. Twelve compound additives were found to substantially suppress methotrexate toxicity.
TK-336 was one of the compound additives found for methotrexate.

| Chemical assayed | % of living flies (n = 50) |
|---|---|
| MTX (.002 mM) | 92 |
| MTX (.3 mM) | 1 |
| MTX (.3 mM) + 336 (1 μM) | 94 |

TK-336 reduces the toxicity of methotrexate by 150-fold in normal fly cells. In addition TK-336 has no toxicity alone. Others compounds along with their fold reduction of methotrexate toxicity in ( ) include:

### TK-618 (150); TK-124 (80); TK-281 (80); TK-403 (80); TK-455 (80); TK-114 (75); TK-108 (75)

### III. Human Cancer Cell Assessment

Methotrexate has been thoroughly demonstrated to have therapeutic effects in a variety of human cancer cell lines. As a quick secondary screen, the additive alone and in combination with the target drug was examined in these human cancer cell lines. The results of TK-336 are shown as a specific example. The compound alone when treated over a wide concentration range had no effects against the cancer cells. Most importantly, when combined with methotrexate, it did not alter the anti-cancer activity of the target drug, also over a large range of additive concentrations.

| | conc./test | | |
|---|---|---|---|
| Cpd | (μg/ml) | cancer cell | Cell survival |
| TK-336 | .64-1.5 | Ovarian | 100% |
| MTX | 30 | Ovarian | 5% |
| MTX | 15 | Ovarian | 50% |
| MTX + TK-336 | 30 + .64 | Ovarian | 5% |

### IV. Mouse Testing

The primary aspect is testing in mice for the ability to translate the toxic reducing action of the additive from flies into mice. Methotrexate was administered at a more moderate dose level, repeatedly over a long period of time. All additives tested for reducing the toxic side effects of MTX in mice were successful. That is 100% of the compounds identified in flies as toxic reducing compounds translated this activity into mice, for both chronic and acute applications.
One of the signs of chronic liver damage by methotrexate is metabolic weight gain. The treatments were done on older juvenile mice (cohort of six). There weight gain was followed for two weeks with treatments given every other day.

| Treatment | Day 0 | Day 7 | Day 14 |
|---|---|---|---|
| None | 110 | 116 | 122 (~5% weight |
| | | | gain/week) |
| Methotrexate | 107 | 107 | 110 (~1% weight |
| | | | gain/week) |
| Methotrexate + TK-336 | 112 | 119 | 126 (~5% weight |
| | | | gain/week) |

At the end of the experiment, livers were examined. The livers of the untreated control were nearly identical in appearance as the ones treated with methotrexate and additive. However, the livers from the methotrexate alone group were smaller, yellow/orange in color and had almost no veins in the organ (5% of the normal density).

It is evident from the above results and discussion that the subject invention provides for methods of reducing the unwanted toxicity of MTX active agents while retaining their desired activity. As such, the subject invention finds use in a variety of different applications and represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of administering to a subject in need thereof an effective amount of an MTX active agent, said method comprising:
administering to said host said effective amount of an MTX active agent in conjunction with an amount of an MTX toxicity reducing agent effective to reduce toxicity of said MTX active agent.

2. The method according to claim 1, wherein said MTX active agent and MTX toxicity reducing agent are administered at the same time.

3. The method according to claim 2, wherein said MTX active agent and MTX toxicity reducing agent are administered as separate formulations.

4. The method according to claim 2, wherein said MTX active agent and MTX toxicity reducing agent are administered in a single formulation.

5. The method according to claim 1, wherein said MTX active agent and said MTX toxicity reducing agent are administered sequentially.

6. The method according to claim 5, wherein said MTX active agent is administered prior to said MTX toxicity reducing agent.

7. The method according to claim 5, wherein said MTX active agent is administered after said MTX toxicity reducing agent.

8. The method according to claim 1, wherein the amount of said MTX toxicity reducing agent is less than the amount of said MTX active agent.

9. The method according to claim 1, wherein said MTX active agent is methotrexate.

10. The method according to claim 1, wherein said MTX toxicity reducing agent is a small organic compound.

11. The method according to claim 10, wherein said small organic compound is chosen from TK-403, TK-191, TK-9791, TK-717, TK-9799, TK-455, TK-678, TK-281, TK-618, TK-406, TK-207, TK-183, TK-743, TK-494, TK-601, TK-280, TK-114, TK-251, TK-128, TK-337, TK-124, TK-108, TK-624, TK-308, TK-222, TK-210, TK-693, TK-398, TK-752 and TK-336.

12. A pharmaceutical composition comprising an effective amount of both an MTX active agent and an MTX toxicity reducing agent in a pharmaceutically acceptable vehicle.

13. The pharmaceutical composition according to claim 12, wherein the amount of said MTX toxicity reducing agent is less than the amount of said MTX active agent.

14. The pharmaceutical composition according to claim 12, wherein said MTX active agent is methotrexate.

15. The pharmaceutical composition according to claim 12, wherein said MTX toxicity reducing agent is a small organic compound.

16. The pharmaceutical composition according to claim 15, wherein said small organic compound is chosen from TK-403, TK-191, TK-9791, TK-717, TK-9799, TK-455, TK-678, TK-281, TK-618, TK-406, TK-207, TK-183, TK-743, TK-494, TK-601, TK-280, TK-114, TK-251, TK-128, TK-337, TK-124, TK-108, TK-624, TK-308, TK-222, TK-210, TK-693, TK-398, TK-752 and TK-336.

17. A method of treating a host suffering from a cellular proliferative disease condition, said method comprising:
administering to said host said effective amount of an MTX active agent in conjunction with an amount of an MTX toxicity reducing agent effective to reduce toxicity of said MTX active agent so that said host is treated for said cellular proliferative disease condition.

18. The method according to claim 17, wherein said MTX active agent and MTX toxicity reducing agent are administered at the same time.

19. The method according to claim 18, wherein said MTX active agent and MTX toxicity reducing agent are administered as separate formulations.

20. The method according to claim 18, wherein said MTX active agent and MTX toxicity reducing agent are administered in a single formulation.

21. The method according to claim 17, wherein said MTX active agent and said MTX toxicity reducing agent are administered sequentially.

22. The method according to claim 21, wherein said MTX active agent is administered prior to said MTX toxicity reducing agent.

23. The method according to claim 21, wherein said MTX active agent is administered after said MTX toxicity reducing agent.

24. The method according to claim 17, wherein the amount of said MTX toxicity reducing agent is less than the amount of said MTX active agent.

25. The method according to claim 17, wherein said MTX active agent is methotrexate.

26. The method according to claim 17, wherein said MTX toxicity reducing agent is a small organic compound.

27. The method according to claim 26, wherein said small organic compound is chosen from TK-403, TK-191, TK-9791, TK-717, TK-9799, TK-455, TK-678, TK-281, TK-618, TK-406, TK-207, TK-183, TK-743, TK-494, TK-601, TK-280, TK-114, TK-251, TK-128, TK-337, TK-124, TK-108, TK-624, TK-308, TK-222, TK-210, TK-693, TK-398, TK-752 and TK-336.

28. A kit for use in treating a host suffering from a cellular proliferative disease condition, said kit comprising:
(a) an MTX active agent; and
(b) an MTX toxicity reducing agent.

29. The kit according to claim 28, wherein said MTX active agent and MTX toxicity reducing agent are present as separate compositions.

30. The kit according to claim 28, wherein said MTX active agent and MTX toxicity reducing agent are present in the same composition.
